(19) United States
(12) Patent Application Publication
(10) Pub. No.: US 2025/0107886 A1
(43) Pub. Date: Apr. 3, 2025

(54) INTRAOCULAR LENSES WITH ADJUSTABLE OPTIC PORTIONS AND METHODS OF POST-OPERATIVELY ADJUSTING INTRAOCULAR LENSES

(71) Applicant: Alcon Inc., Fribourg (CH)

(72) Inventors: Benjamin Michael LEROY, Fort Worth, TX (US); Joselito SORIANO, Hayward, CA (US); Terah Whiting SMILEY, Davis, CA (US); Madison McGOUGH, Fort Worth, TX (US); Matthew Joseph LANOUE, Santa Clara, CA (US); Paul J. MISSEL, Arlington, TX (US)

(73) Assignee: Alcon Inc., Fribourg (CH)

(21) Appl. No.: 18/889,800

(22) Filed: Sep. 19, 2024

Related U.S. Application Data
(60) Provisional application No. 63/586,533, filed on Sep. 29, 2023.

(51) Int. Cl. A61F 2/16

(52) U.S. Cl. CPC A61F 2/1635; A61F 2/1648; A61F 2250/0003

(57) ABSTRACT

Disclosed are adjustable intraocular lenses and methods of adjusting intraocular lenses post-operatively. In one embodiment, an adjustable intraocular lens can comprise an optic portion and one or more haptics. The optic portion can comprise an anterior element, a posterior element, and a fluid-filled optic chamber. At least part of the optic portion can be made of a composite material. The base power of the optic portion can be configured to change in response to an external energy directed at the composite material.

### CROSS-REFERENCE TO RELATED APPLICATION

This application claims priority to U.S. Patent Application No. 63/586,533 filed on Sep. 29, 2023, the content of which is incorporated herein by reference in its entirety.

### TECHNICAL FIELD

The present disclosure relates generally to the field of intraocular lenses, and, more specifically, to intraocular lenses with adjustable optic portions and methods of adjusting such intraocular lenses post-operatively.

### BACKGROUND

A cataract is a condition involving the clouding over of the normally clear lens of a patient's eye. Cataracts occur as a result of aging, hereditary factors, trauma, inflammation, metabolic disorders, or exposure to radiation. Age-related cataract is the most common type of cataracts. In treating a cataract, the surgeon removes the crystalline lens matrix from the patient's lens capsule and replaces it with an intraocular lens (IOL).

However, current IOL surgery may leave some patients unsatisfied with their refractive outcomes. In some cases, the pre-operative biometry measurements made on a patient's eye may be incorrect, leading to IOLs with the wrong lens power being prescribed and implanted within the patient. In other cases, once an IOL is implanted within the capsular bag, an aggressive healing response by tissue within the capsular bag can affect the optical power of the IOL. Moreover, a patient's cornea or muscles within the eye may change as a result of injury, disease, or aging. In such cases, it may also be necessary to adjust the patient's implanted IOLs to account for such changes.

Therefore, a solution is needed which allows for post-implant adjustment of IOLs to address the aforementioned problems without having to undergo additional surgery. Such a solution should not be overly complicated and still allow the IOLs to be cost-effectively manufactured.

### SUMMARY

Disclosed herein are adjustable intraocular lenses and methods of adjusting intraocular lenses post-operatively. In some embodiments, an adjustable intraocular lens is disclosed comprising an optic portion. The optic portion can comprise an anterior element, a posterior element, and an optic chamber defined therebetween. The optic chamber can be filled with a fluid. At least part of the posterior element can be made of a composite material. A base power of the optic portion can be configured to change in response to an external energy directed at the composite material.

In some embodiments, the posterior element can comprise a first posterior portion and a second posterior portion. The first posterior portion and the second posterior portion can be made of the composite material.

In some embodiments, the second posterior portion can be located radially inward from the first posterior portion.

In some embodiments, the second posterior portion can be located posterior of the first posterior portion.

In some embodiments, the base power of the optic portion can be configured to decrease in response to the external energy directed at the first posterior portion.

In some embodiments, the first posterior portion can be configured to expand in response to the external energy directed at the first posterior portion. Expansion of the first posterior portion can increase a volume of the optic chamber.

In some embodiments, the base power of the optic portion can be configured to increase in response to the external energy directed at the second posterior portion.

In some embodiments, the second posterior portion can be configured to expand in response to the external energy directed at the second posterior portion. Expansion of the second posterior portion can decrease a volume of the optic chamber.

In some embodiments, the first posterior portion can be in the form of a first ring-shaped segment. The second posterior portion can be in the form of a second ring-shaped segment.

In some embodiments, the second ring-shaped segment can be smaller in diameter than the first ring-shaped segment.

In some embodiments, the second ring-shaped segment can be positioned concentric with the first ring-shaped segment and radially inward of the first ring-shaped segment.

In some embodiments, the posterior element can comprise an outer posterior surface and an inner posterior surface facing the optic chamber. At least part of the inner posterior surface can serve as a chamber floor of the optic chamber.

In some embodiments, the second posterior portion can be set within the chamber floor.

In some embodiments, at least part of the second posterior portion can be configured to expand in an anterior direction in response to the external energy directed at the second posterior portion.

In some embodiments, a sloped portion of the inner posterior surface can serve as part of a chamber wall of the optic chamber.

In some embodiments, the first posterior portion can be set within the sloped portion of the inner posterior surface.

In some embodiments, at least part of the first posterior portion can be configured to expand in a radially outward direction in response to the external energy directed at the first posterior portion.

In some embodiments, the posterior element further can comprise a raised periphery.

In some embodiments, the first posterior portion can be set within the raised periphery of the posterior element.

In some embodiments, at least part of the first posterior portion can be configured to expand in response to the external energy directed at the first posterior portion. Expansion of the first posterior portion can increase a volume of the optic chamber.

In some embodiments, the anterior element can be bonded or adhered to the posterior element by an adhesive layer. The adhesive layer can also comprise the composite material.

In some embodiments, the adhesive layer can be configured to expand in response to the external energy directed at the adhesive layer. Expansion of the adhesive layer can increase a volume of the optic chamber.

In some embodiments, the base power of the optic portion can be configured to decrease in response to the external energy directed at the adhesive layer.

In some embodiments, the composite material within the posterior element can be configured to expand in response to the external energy directed at the composite material within the posterior element. The expansion of the composite material within the posterior element can decrease the volume of the optic chamber.

In some embodiments, the base power of the optic portion can be configured to increase in response to the external energy directed at the composite material within the posterior element.

In some embodiments, a portion of the posterior element can serve as chamber walls surrounding the optic chamber. The posterior element can further comprise a circular rim extending radially inward from the chamber walls. The circular rim can be made of the composite material.

In some embodiments, at least part of the circular rim can be configured to expand in a radially inward direction in response to the external energy directed at the circular rim.

In some embodiments, expansion of the circular rim can decrease a volume of the optic chamber and increase the base power of the optic portion.

In some embodiments, the composite material can comprise an energy absorbing constituent and a plurality of expandable components.

In some embodiments, the expandable components can be expandable microspheres.

In some embodiments, the energy absorbing constituent can be an energy absorbing colorant.

In some embodiments, the external energy can be laser light having a wavelength of between about 488 nm to about 650 nm.

In some embodiments, the external energy can be laser light having a wavelength of between about 946 nm to about 1120 nm.

In some embodiments, the base power of the optic portion can be configured to change between about 0.05 D to about 3.0 D in total in response to pulses of the external energy directed at the composite material.

In some embodiments, the anterior element can comprise an external optical surface. The external optical surface can comprise a diffractive surface profile or pattern defined on the external optical surface.

In some embodiments, the adjustable intraocular lens can further comprise one or more haptics extending from the optic portion.

In some embodiments, a method of adjusting an intraocular lens post-implantation is disclosed. The method can comprise directing an external energy at a composite material making up part of a posterior element of an optic portion of the intraocular lens. The optic portion can further comprise an anterior element and an optic chamber defined in between the anterior element and the posterior element. The optic chamber can be filled with a fluid. A base power of the optic portion can be configured to change in response to the external energy directed at the composite material. The method can also comprise measuring the change in the base power of the optic portion after directing the external energy at the composite material.

In some embodiments, the posterior element can comprise a first posterior portion and a second posterior portion. The first posterior portion and the second posterior portion can be made of the composite material.

In some embodiments, the second posterior portion can be located radially inward from the first posterior portion.

In some embodiments, the second posterior portion can be located posterior of the first posterior portion.

In some embodiments, the method can further comprise directing the external energy at the first posterior portion in order to decrease the base power of the optic portion.

In some embodiments, the first posterior portion can be configured to expand in response to the external energy directed at the first posterior portion. Expansion of the first posterior portion can increase a volume of the optic chamber.

In some embodiments, the method can further comprise directing the external energy at the second posterior portion in order to increase the base power of the optic portion.

In some embodiments, the second posterior portion can be configured to expand in response to the external energy directed at the second posterior portion. Expansion of the second posterior portion can decrease a volume of the optic chamber.

In some embodiments, the first posterior portion can be in the form of a first ring-shaped segment and the second posterior portion can be in the form of a second ring-shaped segment.

In some embodiments, the second ring-shaped segment can be smaller in diameter than the first ring-shaped segment.

In some embodiments, the second ring-shaped segment can be positioned concentric with the first ring-shaped segment and radially inward of the first ring-shaped segment.

In some embodiments, the posterior element can comprise an outer posterior surface and an inner posterior surface facing the optic chamber. At least part of the inner posterior surface can serve as a chamber floor of the optic chamber.

In some embodiments, the second posterior portion can be set within the chamber floor.

In some embodiments, the method can comprise directing the external energy at the second posterior portion within the chamber floor. At least part of the second posterior portion can be configured to expand in an anterior direction in response to the external energy directed at the second posterior portion within the chamber floor.

In some embodiments, a sloped portion of the inner posterior surface can serve as part of a chamber wall of the optic chamber.

In some embodiments, the first posterior portion can be set within the sloped portion of the inner posterior surface.

In some embodiments, the method can further comprise directing the external energy at the first posterior portion within the sloped portion of the inner posterior surface. At least part of the first posterior portion can be configured to expand in a radially outward direction in response to the external energy directed at the first posterior portion within the sloped portion of the inner posterior surface.

In some embodiments, the posterior element can further comprise a raised periphery.

In some embodiments, the first posterior portion can be set within the raised periphery of the posterior element.

In some embodiments, the method can comprise directing the external energy at the first posterior portion within the raised periphery. At least part of the first posterior portion can be configured to expand in response to the external energy directed at the first posterior portion. Expansion of the first posterior portion can increase a volume of the optic chamber.

In some embodiments, the anterior element can be bonded or adhered to the posterior element by an adhesive layer. The adhesive layer can also comprise the composite material.

In some embodiments, the method can further comprise directing the external energy at the adhesive layer. The adhesive layer can be configured to expand in response to the external energy directed at the adhesive layer. Expansion of the adhesive layer can increase a volume of the optic chamber.

In some embodiments, the base power of the optic portion can be configured to decrease in response to the external energy directed at the adhesive layer.

In some embodiments, the method can further comprise directing the external energy at the composite material within the posterior element. The composite material within the posterior element can be configured to expand in response to the external energy directed thereto. The expansion of the composite material within the posterior element can decrease the volume of the optic chamber.

In some embodiments, the base power of the optic portion can be configured to increase in response to the external energy directed at the composite material within the posterior element.

In some embodiments, a portion of the posterior element can serve as chamber walls surrounding the optic chamber. The posterior element can further comprise a circular rim extending radially inward from the chamber walls. The circular rim can be made of the composite material.

In some embodiments, the method can further comprise directing the external energy at the circular rim. At least part of the circular rim can be configured to expand in a radially inward direction in response to the external energy directed at the circular rim. Expansion of the circular rim can decrease a volume of the optic chamber and increase the base power of the optic portion.

In some embodiments, the composite material can comprise an energy absorbing constituent and a plurality of expandable components.

In some embodiments, the expandable components can be expandable microspheres.

In some embodiments, the energy absorbing constituent can be an energy absorbing colorant.

In some embodiments, the external energy can be laser light having a wavelength of between about 488 nm to about 650 nm.

In some embodiments, the external energy can be laser light having a wavelength of between about 946 nm to about 1120 nm.

In some embodiments, the base power of the optic portion can be configured to change between about 0.05 D to about 3.0 D in total in response to pulses of the external energy directed at the composite material.

In some embodiments, the anterior element can comprise an external optical surface. The external optical surface can comprise a diffractive surface profile or pattern defined on the external optical surface.

In some embodiments, the intraocular lens can further comprise one or more haptics extending from the optic portion.

In some embodiments, an adjustable intraocular lens is disclosed comprising an optic portion comprising an optic chamber filled with a fluid. The optic chamber can comprise a chamber floor and chamber walls. At least part of the chamber floor can be made of a composite material. A base power of the optic portion can be configured to change in response to an external energy directed at the chamber floor.

In some embodiments, the base power of the optic portion can be configured to increase in response to the external energy directed at the chamber floor.

In some embodiments, at least part of the chamber walls can be made of the composite material. The base power of the optic portion can be configured to change in response to an external energy directed at the chamber walls.

In some embodiments, the base power of the optic portion can be configured to decrease in response to the external energy directed at the chamber walls.

In some embodiments, an adjustable intraocular lens is disclosed comprising an optic portion comprising an optic chamber filled with a fluid. At least part of the optic chamber can be made of a composite material. A base power of the optic portion can be configured to change in response to an external energy directed at the composite material.

In some embodiments, the optic portion can comprise a first optic portion and a second optic portion. The first optic portion and the second optic portion can be made of the composite material.

In some embodiments, the second optic portion can be located radially inward from the first optic portion.

In some embodiments, the second optic portion can be located posterior of the first optic portion.

In some embodiments, the base power of the optic portion can be configured to decrease in response to the external energy directed at the first optic portion.

In some embodiments, the first optic portion can be configured to expand in response to the external energy directed at the first optic portion. Expansion of the first optic portion can increase a volume of the optic chamber.

In some embodiments, the base power of the optic portion can be configured to increase in response to the external energy directed at the second optic portion. The second optic portion can be configured to expand in response to the external energy directed at the second optic portion, and wherein expansion of the second optic portion decreases a volume of the optic chamber.

In some embodiments, the first optic portion can be in the form of a first ring-shaped segment. The second optic portion can be in the form of a second ring-shaped segment.

In some embodiments, the second ring-shaped segment can be smaller in diameter than the first ring-shaped segment.

In some embodiments, the second ring-shaped segment can be positioned concentric with the first ring-shaped segment and radially inward of the first ring-shaped segment.

In some embodiments, the optic portion can comprise a chamber floor and chamber walls. The second optic portion can be set within the chamber floor. At least part of the second optic portion can be configured to expand in an anterior direction in response to the external energy directed at the second optic portion.

In some embodiments, at least a portion of the chamber wall can be sloped. The first optic portion can be set within the sloped portion of the chamber wall. At least part of the first optic portion can be configured to expand in a radially outward direction in response to the external energy directed at the first optic portion. Expansion of the first optic portion can increase a volume of the optic chamber.

In some embodiments, the optic portion can comprise an adhesive layer. The adhesive layer can comprise the composite material. The adhesive layer can be configured to expand in response to the external energy directed at the adhesive layer. Expansion of the adhesive layer can increase a volume of the optic chamber. The base power of the optic portion can be configured to decrease in response to the external energy directed at the adhesive layer.

In some embodiments, the optic portion can comprise chamber walls surrounding the optic chamber. The optic portion can comprise a circular rim extending radially inward from the chamber walls. The circular rim can be made of the composite material. At least part of the circular rim can be configured to expand in a radially inward direction in response to the external energy directed at the circular rim. Expansion of the circular rim can decrease a volume of the optic chamber and can increase the base power of the optic portion.

In some embodiments, the composite material can comprise an energy absorbing constituent and a plurality of expandable components.

In some embodiments, the expandable components can be expandable microspheres.

In some embodiments, the energy absorbing constituent can be an energy absorbing colorant.

In some embodiments, the external energy can be laser light having a wavelength of between about 488 nm to about 650 nm.

In some embodiments, the external energy can be laser light having a wavelength of between about 946 nm to about 1120 nm.

In some embodiments, the base power of the optic portion can be configured to change between about 0.05 D to about 3.0 D in total in response to pulses of the external energy directed at the composite material.

In some embodiments, the optic portion can comprise an external optical surface. The external optical surface can comprise a diffractive surface profile or pattern defined on the external optical surface.

In some embodiments, the adjustable intraocular lens can also comprise one or more haptics extending from the optic portion.

### BRIEF DESCRIPTION OF THE DRAWINGS

FIG. 1 illustrates an exploded perspective view of one embodiment of an adjustable intraocular lens (IOL).

FIG. 2A illustrates a composite material used to make at least part of the adjustable IOL.

FIG. 2B illustrates one embodiment of an expandable component of the composite material.

FIG. 3 illustrates a cross-sectional view of an optic portion of one embodiment of the adjustable IOL.

FIG. 4A is a black-and-white image of an optic portion of one embodiment of the adjustable IOL after pulses of laser light (the external energy in this case) have been directed at the composite material making up a part of the optic portion.

FIG. 4B is a black-and-white image of an optic portion of another embodiment of the adjustable IOL after pulses of laser light (the external energy in this case) have been directed at the composite material making up part of the optic portion.

FIG. 4C is a close-up black-and-white image showing an activated composite material expanding into an optic chamber in response to an external energy having been directed at the composite material.

FIG. 4D is a computed tomography (CT) scan showing an activated composite material making up part of a chamber wall surrounding an optic chamber.

FIG. 5 illustrates a cross-sectional view of the optic portion of another embodiment of the adjustable IOL.

FIG. 6 is an image showing the results of a finite element analysis (FEA) performed on an embodiment of the adjustable IOL after pulses of laser light are directed at the composite material of the adjustable IOL.

FIG. 7 illustrates a cross-sectional view of the optic portion of yet another embodiment of the adjustable IOL where an adhesive layer comprises the composite material.

FIG. 8 is an image showing the results of a finite element analysis (FEA) performed on the adjustable IOL of FIG. 7 after pulses of laser light are directed at an adhesive layer made in part of the composite material.

FIG. 9A illustrates a cross-sectional view of the optic portion of one embodiment of the adjustable IOL with the composite material extending radially inward from a chamber wall of the adjustable IOL.

FIG. 9B illustrates a cross-sectional view of the optic portion of the adjustable IOL of FIG. 9A after pulses of laser light are directed at the composite material extending radially inward from the chamber wall of the adjustable IOL.

FIGS. 10A-10C illustrate various methods of adjusting an IOL post-operatively or post-implantation.

### DETAILED DESCRIPTION

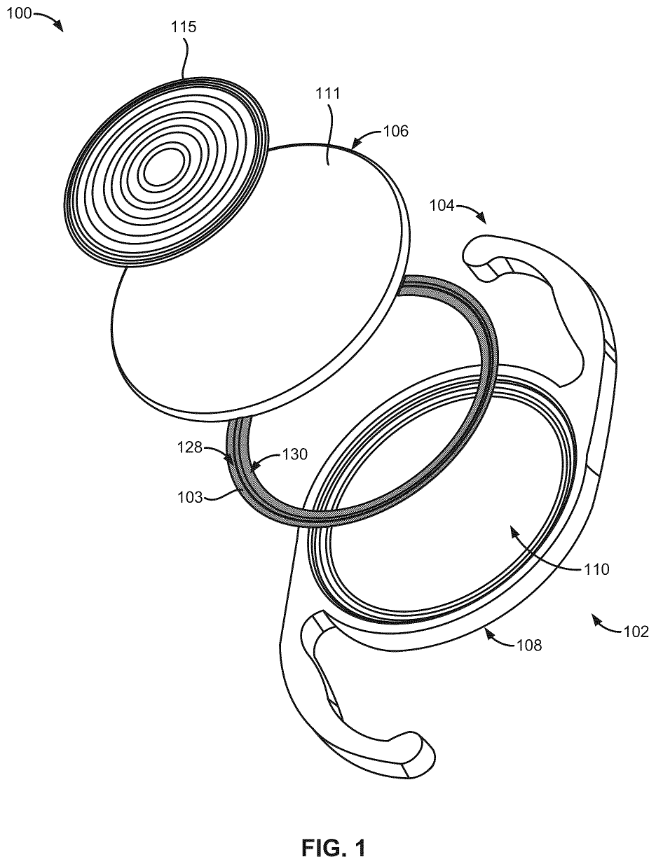

FIG. 1 illustrates an exploded perspective view of one embodiment of an adjustable intraocular lens (IOL) 100. As depicted in FIG. 1, the adjustable IOL 100 can comprise an optic portion 102 and one or more haptics 104 extending from the optic portion 102.

The haptics 104 can comprise a first haptic and a second haptic extending peripherally from or coupled to the optic portion 102. Each of the haptics 104 can comprise a kink or bend defined along an arm of the haptic 104. The kink or bend can allow the haptic 104 to compress or flex in response to capsular bag reshaping. Each of the haptics 104 can terminate at a free or unconnected haptic distal end.

For example, the adjustable IOL 100 can be a one-piece lens such that the haptics 104 are connected to and extends from the optic portion 102. In this example embodiment, the haptics 104 are formed along with the optic portion 102 and is not adhered or otherwise coupled to the optic portion 102 in a subsequent step.

In other embodiments, the haptics 104 are coupled to and adhered to the optic portion 102. For example, the haptics 104 can be adhered to the optic portion 102 after each is formed separately.

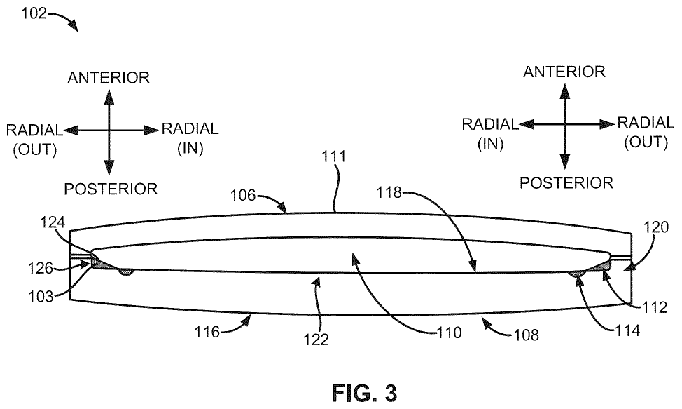

The optic portion 102 can comprise an anterior element 106, a posterior element 108, and an optic chamber 110 defined in between the anterior element 106 and the posterior element 108. The optic chamber 110 (see also, e.g., FIGS. 3, 5, 7, 9A, and 9B) can be filled with a fluid.

In some embodiments, the fluid within the optic chamber 110 can be an oil. More specifically, in certain embodiments, the fluid within the optic chamber 110 can be a silicone oil.

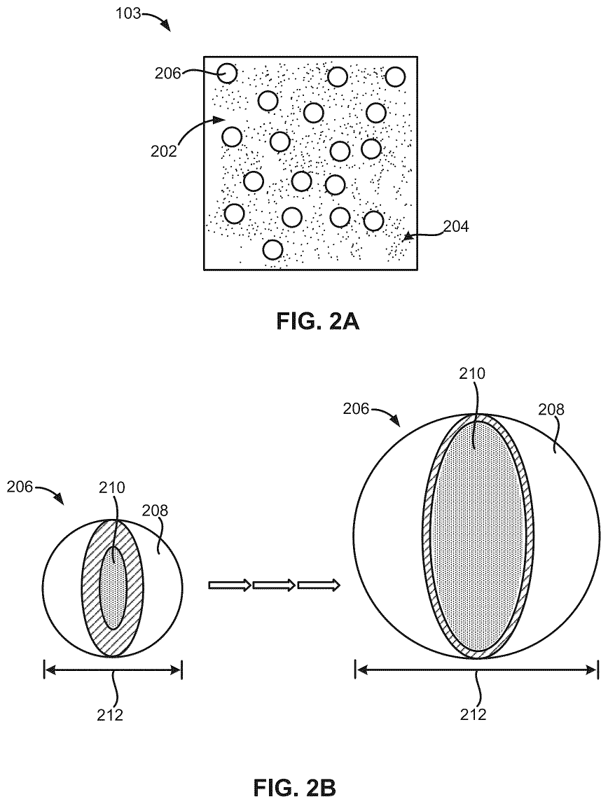

At least part of the posterior element 108 can be made of a composite material 103 or comprise the composite material 103. As will be discussed in more detail in the following sections, the composite material 103 can comprise an energy absorbing constituent 204 and a plurality of expandable components 206 (see, e.g., FIGS. 2A and 2B).

As shown in FIG. 1, in some embodiments, the composite material 103 can be formed as an annulus or a plurality of concentric rings (where the concentric rings are connected or unconnected to one another).

In certain embodiments, parts of the posterior element 108 can be shaped or otherwise formed (e.g., by a lathe tool or cutting tool) to accommodate the composite material 103. In these and other embodiments, the composite material 103 can be coupled or adhered to the rest of the posterior element 108. For example, one or more circular trenches or hollowed-out portions can be formed along an anterior portion of the posterior element 108. In this example, the trench(es) or hollowed-out portion(s) can be filled in with the composite material 103.

In some embodiments, the posterior element 108 can be cast and cured alongside the composite material 103. In these embodiments, the composite material 103 can be integrated with the posterior element 108 or formed along within the posterior element 108.

As will be discussed in more detail in later sections, the composite material 103 can be configured to expand in response to an external energy directed at or otherwise applied to the composite material 103. Expansion of the composite material 103 can cause a change in a volume of the optic chamber 110 (or an available volume for holding the fluid within the optic chamber 110).

Depending on where the composite material 103 is placed or positioned within the posterior element 108, expansion of the composite material 103 can either increase or decrease the volume of the optic chamber 110.

Also, as will be discussed in more detail in later sections, a base power of the optic portion can be configured to change in response to any changes in the volume of the optic chamber 110 caused by the external energy directed at the composite material 103. For example, the anterior element 106 of the optic portion 102 can flex or deform (or otherwise change shape or change its curvature) in response to a change in the fluid pressure within the optic chamber 110 due to a change in the volume of the optic chamber 110. In certain embodiments, at least part of the posterior element 108 can also flex or deform (or otherwise change shape or change its curvature) in response to a change in the fluid pressure within the optic chamber 110.

A base power or optical/dioptric power of the optic portion 102 can be configured to change when the anterior element 106 and/or the posterior element 108 flexes or deforms in response to the change in the fluid pressure within the optic chamber 110 caused by the external energy directed at the composite material 103.

In some embodiments, directing an external energy at one portion of the posterior element 108 made of the composite material 103 can cause that particular portion of the posterior element 108 to change its shape or expand without substantially affecting the other portions of the posterior element 108.

Moreover, as will be discussed in more detail in the following sections, directing pulses of the external energy at one portion of the posterior element 108 can cause a change in the base power of the optic portion 102 in one direction (e.g., an increase in the base power). In these embodiments, directing additional pulses of the external energy at another portion of the posterior element 108 can cause a change in the base power of the optic portion 102 in another direction (e.g., a decrease in the base power).

In some embodiments, the base power of the optic portion 102 can be configured to change in total between about 0.05 diopter (D) up to about 3.0 D (e.g., about 2.0 D) in either a positive or negative direction in response to the external energy (e.g., pulses of laser light) directed at the composite material 103 within the optic portion 102.

The change in the base power of the optic portion 102 can be a persistent or a substantially permanent change. A persistent or substantially permanent change can mean that the composite material 103 does not substantially revert back to its original shape or size after the change has occurred.

In some embodiments, the optic portion 102 can have an unfilled or as-manufactured optical power (i.e., an optical power of the optic portion 102 when the optic chamber 110 is empty or unfilled) of between about 11 D and 13 D (a “zero power” lens). For example, the optic portion 102 can have an unfilled or as-manufactured optical power of about 12 D. The optical power of the optic portion 102 can increase as the optic chamber 110 is filled with the fluid (e.g., the silicone oil).

The optic chamber 110 can be filled until the base power of the filled optic portion 102 (as contributed by both the fluid and the lens surfaces of the optic portion 102) is between about 15 D (a low-powered IOL) to about 30 D (a high-powered IOL). For example, the optic chamber 110 can be filled until the base power of the filled optic portion 102 is about 20 D.

The adjustable IOL 100 implanted within a capsular bag of the subject can have a base power between about 15 D to about 30 D (e.g., about 20 D). A clinician or medical professional can direct the external energy (e.g., pulses of laser light) at the composite material 103 within the optic portion 102 to increase or decrease the base power of the optic portion when the adjustable IOL 100 is implanted within the capsular bag of the subject.

For example, the adjustable IOL 100 can have a base power of about 20 D when implanted within the eye of the subject. If power correction is desired to increase the power of the lens, a clinician or medical professional can direct the external energy at the composite material 103 making up part of the optic portion 102 to increase the base power of the optic portion 102 until the final base power is at a desired base power.

As another example, the adjustable IOL 100 can have a base power of about 25 D when implanted within the eye of the subject. If power correction is desired to decrease the power of the lens, a clinician or medical professional can direct the external energy at another instance of the composite material 103 making up part of the optic portion 102 to decrease the base power of the optic portion 102 until the final base power is at a desired base power.

In some embodiments, the adjustable IOL 100 can be designed or configured such that the base power of the optic portion 102 can be unresponsive or insensitive to forces applied to the one or more haptics 104 by the capsular bag when the adjustable IOL 100 is implanted within the capsular bag.

In some embodiments, the external energy can be light energy. More specifically, the external energy can be laser light. The external energy can be bursts or pulses of laser light.

In certain embodiments, the laser light can have a wavelength between about 488 nm to about 650 nm. For example, the laser light can be green laser light. The green laser light can have a wavelength of between about 520 nm to about 570 nm. In one example, embodiment, the external energy can be green laser light having a wavelength of about 532 nm.

For example, the laser light can be laser light emitted by an ophthalmic laser. For example, the laser light can be laser light emitted by a retinal coagulation laser.

In certain embodiments, the laser light can be emitted by a neodymium-doped yttrium aluminum garnet (Nd:YAG) laser. As a more specific example, the laser light can be a pulsed Nd:YAG laser operating in a Q-switching mode and frequency doubled to generate laser light at 532 nm.

In other embodiments, the laser light can be emitted by a femtosecond laser or an infrared or near infrared laser. For example, the laser light emitted by such lasers can have a wavelength of between about 1030 nm and 1064 nm.

As will be discussed in more detail in the following sections, when the external energy is light energy, energy absorbing constituents (see FIG. 2A) within the composite material 103 can absorb or otherwise capture the light energy and convert the light energy into thermal energy and transfer the thermal energy to expandable components (see FIGS. 2A and 2B) within the composite material 103 to expand the expandable components.

FIG. 1 also illustrates that the anterior element 106 can comprise an external optical surface 111. The external optical surface 111 can comprise a unique lens surface profile 115 or pattern defined on the external optical surface 111.

In some embodiments, the lens surface profile 115 can comprise a central diffractive area or structure comprising a plurality of diffractive zones or steps. In these and other embodiments, the widths of the diffractive zones can decrease in a radially outward manner such that zone widths at a periphery of the lens are smaller than zone widths near a central portion of the lens.

In certain embodiments, the lens surface profile 115 can split light into multiple foci or focal points. In these embodiments, the adjustable IOL 100 can be considered an adjustable multifocal IOL.

In some embodiments, the lens surface profile 115 can be configured to split light into two focal points (e.g., allowing for near and distant vision). In these embodiments, the adjustable IOL 100 can be considered an adjustable bifocal IOL.

The lens surface profile 115 can also be configured to split light into three focal points (e.g., allowing for near, intermediate, and distant vision). In these embodiments, the adjustable IOL 100 can be considered an adjustable trifocal IOL.

In other embodiments not shown in FIG. 1, the external optical surface 111 can have a uniformly curved (e.g., a spherical) lens surface or an aspherical lens surface providing focusing power for a single distance. In these embodiments, the adjustable IOL 100 can be considered an adjustable monofocal IOL.

In additional embodiments not shown in FIG. 1, the external optical surface 111 can have a lens surface profile or pattern configured to provide an extended depth of focus or a single elongated focal point. In these embodiments, the adjustable IOL 100 can be considered an adjustable extended depth of focus (EDOF) IOL or a non-accommodating fluid-adjustable EDOF IOL.

Moreover, any of the adjustable monofocal IOL, the adjustable multifocal IOL, and the adjustable EDOF IOL can comprise a toric lens profile.

One technical problem faced by the applicant is how to design an adjustable fluid-filled IOL that can be used by patients seeking different types of vision support (e.g., near vision, intermediate vision, distance vision, etc.). One solution discovered by the applicant is the adjustable IOL disclosed herein where different lens surface profiles, both rotationally symmetric as well as toric profiles, can be defined on the external optical surface 111 of the optic portion 102 allowing for the same adjustable IOL structure to be adapted as an adjustable monofocal IOL, an adjustable bifocal IOL, an adjustable trifocal IOL, or an adjustable EDOF IOL, in both toric and non-toric shapes.

The adjustable IOL 100 can have an uncompressed haptic length as measured from a haptic distal end of the first haptic to the haptic distal end of the second haptic. The uncompressed haptic length can be between about 12.0 mm and about 14.0 mm. For example, the uncompressed haptic length can be about 13.0 mm.

In some embodiments, the optic portion 102 can have an optic portion diameter. The optic portion diameter can be between about 5.0 mm and 8.0 mm. For example, the optic portion diameter can be about 6.0 mm.

In some embodiments, the parts of the optic portion 102 not made of the composite material 103 can comprise or be made in part of a lens body material. The lens body material can be made in part of a cross-linked copolymer comprising a copolymer blend. The copolymer blend can comprise an alkyl acrylate or methacrylate, a fluoro-alkyl (meth)acrylate, and a phenyl-alkyl acrylate. It is contemplated by this disclosure and it should be understood by one of ordinary skill in the art that these types of acrylic cross-linked copolymers can be generally copolymers of a plurality of acrylates, methacrylates, or a combination thereof and the term “acrylate” as used herein can be understood to mean acrylates, methacrylates, or a combination thereof interchangeably unless otherwise specified.

The cross-linked copolymer used to make the lens body material can comprise an alkyl acrylate or methacrylate in the amount of about 3% to 20% (wt %), a fluoro-alkyl acrylate or fluoro-alkyl methacrylate in the amount of about 10% to 35% (wt %), and a phenyl-alkyl acrylate in the amount of about 50% to 80% (wt %). In some embodiments, the cross-linked copolymer can comprise or be made in part of an n-butyl acrylate as the alkyl acrylate, trifluoroethyl methacrylate as the fluoro-alkyl acrylate, and phenylethyl acrylate as the phenyl-alkyl acrylate. More specifically, the cross-linked copolymer used to make the lens body material can comprise n-butyl acrylate in the amount of about 3% to 20% (wt %) (e.g., between about 12% to 16%), trifluoroethyl methacrylate in the amount of about 10% to 35% (wt %) (e.g., between about 17% to 25%), and phenylethyl acrylate in the amount of about 50% to 80% (wt %) (e.g., between about 64% to 67%).

The final composition of the cross-linked copolymer used to make the lens body material can also comprise a cross-linker or cross-linking agent, such as ethylene glycol dimethacrylate (EGDMA), and a hydroxyl-functional acrylic monomer (hydroxyethyl acrylate (HEA) or hydroxyethyl methacrylate (HEMA)). For example, the final composition of the cross-linked copolymer used to make the lens body material can also comprise a cross-linker or cross-linking agent (e.g., EGDMA) in the amount of about 1.0%. The final composition of the cross-linked copolymer used to make the lens body material can also comprise an initiator or initiating agent (e.g., Perkadox 16, Darocur, etc.) and a UV absorber.

The haptic(s) 104 can comprise or be made in part of a haptic material. The haptic material can comprise or be made in part of a cross-linked copolymer comprising a copolymer blend. The copolymer blend can comprise an alkyl acrylate, a fluoro-alkyl acrylate, and a phenyl-alkyl acrylate. For example, the cross-linked copolymer used to make the haptic material can comprise an alkyl acrylate in the amount of about 10% to 25% (wt %), a fluoro-alkyl acrylate in the amount of about 10% to 35% (wt %), and a phenyl-alkyl acrylate in the amount of about 50% to 80% (wt %). In some embodiments, the cross-linked copolymer used to make the haptic material can comprise n-butyl acrylate in the amount of about 10% to 25% (wt %) (e.g., between about 19% to about 23%), trifluoroethyl methacrylate in the amount of about 10% to 35% (wt %) (e.g., between about 14% to about 18%), and phenylethyl acrylate in the amount of about 50% to 80% (wt %) (e.g., between about 58% to about 62%). The final composition of the cross-linked copolymer used to make the haptic material can also comprise a cross-linker or cross-linking agent, such as EGDMA, in the amount of about 1.0%. The final composition of the cross-linked copolymer used to make the haptic material can also comprise a number of photoinitiators or photoinitiating agents (e.g., camphorquinone, 1-phenyl-1,2-propanedione, 2-ethylhexyl-4-(dimenthylamino)benzoate, etc.).

In some embodiments, the refractive index of the lens body material can be between about 1.48 and about 1.53. In certain embodiments, the refractive index of the lens body material can be between about 1.50 and about 1.53 (e.g., about 1.5178).

The optic portion 102 can be configured to deform, flex, or otherwise change shape in response to a change in the fluid pressure within the optic chamber 110. The fluid pressure within the optic chamber 101 can change due to the volume of the optic chamber 110 changing due to the expansion of the composite material 103.

As previously discussed, the fluid within the optic chamber 110 can be a silicone oil. In some embodiments, the silicone oil can comprise or be made in part of a ratio of two dimethyl siloxane units to one diphenyl siloxane unit. In certain embodiments, the silicone oil can comprise about 20 mol % diphenyl siloxane and about 80 mol % dimethyl siloxane.

More specifically, in some embodiments, the silicone oil can comprise diphenyltetramethyl cyclotrisiloxane. In additional embodiments, the silicone oil can comprise or be made in part of a diphenyl siloxane and dimethyl siloxane copolymer.

The fluid (e.g., the silicone oil) can be index matched with the lens body material used to make the optic portion 102. When the fluid is index matched with the lens body material, the entire optic portion 102 containing the fluid acts as a single lens. For example, the fluid can be selected so that it has a refractive index of between about 1.48 and 1.53 (or between about 1.50 and 1.53).

In some embodiments, the fluid (e.g., the silicone oil) can have a polydispersity index of between about 1.2 and 1.3. In other embodiments, the fluid (e.g., the silicone oil) can have a polydispersity index of between about 1.3 and 1.5. In other embodiments, the fluid (e.g., the silicone oil) can have a polydispersity index of between about 1.1 and 1.2. Other example fluids are described in U.S. Pat. No. 8,900,298, the content of which is incorporated herein by reference in its entirety.

The adjustable IOL 100 can be implanted within a native capsular bag in which a native lens has been removed. When implanted within the native capsular bag, the optic portion 102 can be adapted to refract light that enters the eye onto the retina. The one or more haptics 104 can be configured to engage the capsular bag to hold the adjustable IOL 100 in place within the capsular bag.

FIG. 2A is a graphic representation of a composite material 103 comprising a composite base material 202, an energy absorbing constituent 204, and a plurality of expandable components 206. As previously discussed, one or more portions of the optic portion 102 can be made of the composite material 103.

The composite base material 202 can be comprised of hydrophobic acrylic materials. For example, the composite base material 202 can be comprised of phenylethyl acrylate (PEA), a phenylethyl methacrylate (PEMA), or a combination thereof.

In one example embodiment, the composite base material 202 can comprise a methacrylate-functional or methacrylic-functional cross-linkable polymer and reactive acrylic monomer diluents including lauryl methacrylate (n-dodecyl methacrylate or SR313) and ADMA. By controlling the amount of lauryl methacrylate (SR313) to ADMA, the overall corresponding hardness (i.e., more ADMA) or softness (i.e., more SR313) of the cured composite material 103 can be controlled. The methacrylate-functional or methacrylic-functional cross-linkable polymer can be made using the cross-linkable polymer precursor formulation.

The cross-linkable polymer precursor formulation can comprise the same copolymer blend used to make the optic portion 102 and the haptics 104.

The copolymer blend can comprise an alkyl acrylate or methacrylate (e.g., n-butyl acrylate), a fluoro-alkyl (meth)acrylate (e.g., trifluoroethyl methacrylate), and a phenyl-alkyl acrylate (e.g., phenylethyl acrylate). For example, the copolymer blend can comprise n-butyl acrylate in the amount of about 41% to about 45% (wt %), trifluoroethyl methacrylate in the amount of about 20% to about 24% (wt %), and phenylethyl acrylate in the amount of about 28% to about 32% (wt %). The cross-linkable polymer precursor formulation can comprise or be made in part of the copolymer blend, a hydroxyl-functional acrylic monomer (e.g., HEA), and a photoinitiator (e.g., Darocur 4265 or a 50/50 blend of diphenyl(2,4,6-trimethylbenzoyl)-phosphine oxide and 2-hydroxy2-methylpropiophenone).

The composite base material 202 can comprise the methacrylate-functional or methacrylic-functional cross-linkable polymer (as discussed above) in the amount of about 50% to about 65% (e.g., about 55% to about 60%) (wt %), the reactive acrylic monomer diluent lauryl methacrylate (SR313) in the amount of about 32% to about 38% (e.g., about 32.70%) (wt %), the reactive acrylic monomer diluent adamantly methacrylate (ADMA) in the amount of about 5% to about 9% (e.g., about 7.30%) (wt %).

Table 1 below provides an example formulation for the composite material 103:

TABLE 1
FORMULATION OF COMPOSITE MATERIAL (WT %)

| | |
|---|---|
| Cross-linkable polymer (in | 1.47% 2-hydroxyethyl acrylate (HEA) |
| two steps from precursor | 1.96% Darocur 4265 (photoinitiator) |
| formulation, as described | 43.49% n-butylacrylate (nBA) |
| above) | 30.21% 2-phenylethylacrylate (PEA) |
| | 22.87% 2,2,2-trifluoroethylmethacrylate |
| | (TFEMA) |
| Composite base material | 60.00% cross-linkable polymer |
| | 32.70% lauryl methacrylate (SR313) |
| | 7.30% 1-adamantyl methacrylate (ADMA) |
| Composite base material with | 99.50% composite base material |
| red energy absorbing colorant | 0.50% Disperse Red 1 dye |
| Composite base material with | 99.95% composite base material |
| black energy absorbing | 0.05% graphitized mesoporous carbon |
| colorant | black |
| Final formulation of | 87.70% composite base material with red |
| composite material | or black energy absorbing colorant |
| | 10.00% expandable microspheres |
| | 1.00% Luperox peroxide (thermal |
| | initiator) |
| | 1.30% Omnirad 2022 |

The composite material 103 can be made in several operations. The first operation can comprise preparing an uncolored composite base material 202. The second operation can comprise mixing the composite base material 202 with an energy absorbing constituent 204, expandable components 206, and initiators such as one or more photoinitiators, thermal initiators, or a combination thereof. The third operation can comprise placing the uncured composite material 103 into a desired location within the optic portion 102 and curing the composite material 103 into place.

For example, the uncolored composite base material 202 can be mixed with an energy absorbing constituent 204 such as a dye (e.g., Disperse Red 1 dye) or pigment (graphitized carbon black). The energy absorbing constituent 204 will be discussed in more detail below.

In some embodiments, the expandable components 206 can make up about 5.0% to about 15.0% by weight of a final formulation of the composite material 103. More specifically, the expandable components 206 can make up about 8.0% to about 12.0% (e.g., about 10.0%) by weight of a final formulation (see Table 1) of the composite material 103. In these and other embodiments, the energy absorbing constituent 204 can make up about 0.044% to about 0.44% (or about 0.55%) by weight of the final formulation of the composite material 103.

The photoinitiator can be Omnirad 2022 (bis(2,4,6-trimethylbenzoyl)phenyl-phosphineoxide/2-hydroxy-2-methyl-1-phenyl-propan-1-one). The photoinitiator can make up about 1.30% by weight of a final formulation of the composite material 103 (see, e.g., Table 1). In addition, the composite material 103 can also comprise a thermal initiator. The thermal initiator can make up about 1.00% by weight of a final formulation of the composite material 103 (see, e.g., Table 1). In some embodiments, the thermal initiator can be a dialkyl peroxide such as Luperox® peroxide. In other embodiments, the thermal initiator can be Perkadox.

In some embodiments, the energy absorbing constituent 204 can absorb the external energy (e.g., laser energy), convert the energy to heat, and conduct the energy to the composite base material 202 to expand the composite base material 202.

FIG. 2B illustrates that the expandable components 206 can be expandable microspheres comprising an expandable thermoplastic shell 208 and a blowing agent 210 contained within the expandable thermoplastic shell 208. The microspheres can be configured to expand such that a diameter 212 of at least one of the microspheres can increase about 2× the original diameter. In other embodiments, the microspheres can be configured to expand such that the diameter 212 of at least one of the microspheres can increase about 4× or four times the original diameter. In further embodiments, the microspheres can be configured to expand such that the diameter 212 of at least one of the microspheres can increase between about 2× and about 4× (or about 3.5×) the original diameter. For example, the microspheres can have a diameter 212 of about 12 μm at the outset. In response to an external energy applied or directed at the composite material 103 or in response to energy transferred or transmitted to the microspheres, the diameter 212 of the microspheres can increase to about 40 μm.

The volume of at least one of the microspheres can be configured to expand between about ten times (10×) to about 50 times (50×) in response to the external energy applied or directed at the composite material 103 or in response to energy transferred or transmitted to the microspheres.

In some embodiments, the blowing agent 210 can be an expandable fluid, such as an expandable gas. More specifically, the blowing agent 210 can be a branched-chain hydrocarbon. For example, the blowing agent 210 can be isopentane. In other embodiments, the blowing agent 210 can be or comprise cyclopentane, pentane, or a mixture of cyclopentane, pentane, and isopentane.

The expandable components 206 can comprise differing amounts of the blowing agent 210. For example, some expandable components 206 can comprise more or a greater amount of the blowing agent (e.g., more expandable gas) to allow such expandable components 206 to expand more, resulting in greater expansion of the composite material 103 comprising such expandable components 206.

FIG. 2B illustrates that each of the expandable components 206 can comprise a thermoplastic shell 208. FIG. 2B also illustrates that a thickness of the thermoplastic shell 208 can change as the expandable component 206 increases in size. More specifically, the thickness of the thermoplastic shell 208 can decrease as the expandable component 206 increases in size. For example, when the expandable components 206 are expandable microspheres, the thickness of the thermoplastic shell 208 (i.e., its thickness in a radial direction) can decrease as the diameter 212 of the expandable microsphere increases.

For example, as previously discussed, at least one of the expandable microspheres can have a diameter 212 of about 12 μm at the outset. In this embodiment, the thermoplastic shell 208 of the expandable microsphere can have a shell thickness of about 2.0 μm. In response to an external energy applied or directed at the composite material 103 or in response to energy transferred or transmitted to the microsphere, the diameter 212 of the microsphere can increase to about 40 μm (and the volume expand between about 10× and 50×) and the shell thickness of the microsphere can decrease to about 0.1 μm.

Although FIGS. 2A and 2B illustrate the expandable components 206 as spheres or microspheres, it is contemplated by this disclosure that the expandable components 206 can be substantially shaped as ovoids, ellipsoids, cuboids or other polyhedrons, or a combination thereof.

In some embodiments, the thermoplastic shell 208 can be made in part of nitriles or acrylonitrile copolymers. For example, the thermoplastic shell 208 can be made in part of acrylonitrile, styrene, butadiene, methyl acrylate, or a combination thereof.

As previously discussed, the expandable components 206 can make up between about 8.0% to about 12% by weight of a final formulation of the composite material 103. The expandable components 206 can make up about 10% by weight of a final formulation of the composite material 103.

The expandable components 206 can be dispersed or otherwise distributed within the composite base material 202 making up the bulk of the composite material 103. The composite base material 202 can serve as a matrix for holding or carrying the expandable components 206. The composite material 103 can expand in response to an expansion of the expandable components 206 (e.g., the thermoplastic microspheres). For example, a volume of the composite material 103 can be configured to increase in response to the expansion of the expandable components 206.

The composite material 103 also comprises an energy absorbing constituent 204. In some embodiments, the energy absorbing constituent 204 can be an energy absorbing colorant.

In certain embodiments, the energy absorbing colorant can be an energy absorbing dye. For example, the energy absorbing dye can be an azo dye. In some embodiments, the azo dye can be a red azo dye such as Disperse Red 1 dye. In other embodiments, the azo dye can be an orange azo dye such as Disperse Orange dye (e.g., Disperse Orange 1), a yellow azo dye such as Disperse Yellow dye (e.g., Disperse Yellow 1), a blue azo dye such as Disperse Blue dye (e.g., Disperse Blue 1), or a combination thereof.

In additional embodiments, the energy absorbing colorant can be or comprise a pigment. For example, the energy absorbing colorant can be or comprise graphitized carbon black as the pigment.

Similar to the expandable components 206, the energy absorbing constituent 204 can be dispersed or otherwise distributed within the composite base material 202 making up the bulk of the composite material 103. The composite base material 202 can serve as a matrix for holding or carrying the expandable components 206 and the energy absorbing constituent 204.

As previously discussed, the energy absorbing constituent 204 can make up between about 0.025% to about 1.0% (or, more specifically, about 0.045% to about 0.45%) by weight of a final formulation of the composite material 103. For example, when the energy absorbing constituent 204 is a dye (e.g., an azo dye such as Disperse Red 1), the energy absorbing constituent 204 can make up about between about 0.45% to about 1.0% by weight of a final formulation of the composite material 103. When the energy absorbing constituent 204 is graphitized carbon black or other types of pigments, the energy absorbing constituent 204 can make up about 0.025% to about 0.045% by weight of a final formulation of the composite material 103.

The energy absorbing constituent 204 (e.g., azo dye, graphitized carbon black, or a combination thereof) can absorb or capture an external energy (e.g., light energy or, more specifically, laser light) applied or directed at the composite material 103. The energy absorbing constituent 204 can absorb or capture the external energy and then transform or transfer the energy into thermal energy or heat to the expandable components 206.

The thermoplastic shell 208 can soften and begin to flow as thermal energy is transferred or transmitted to the expandable components 206. The thermoplastic shell 208 of the expandable components 206 can then begin to thin or reduce in thickness in response to the thermal energy transferred or transmitted to the expandable components 206. As the thermoplastic shell 208 begins to soften and reduce in thickness, the blowing agent 210 within the expandable components 206 can expand. The blowing agent 210 can also expand in response to the thermal energy or heat transferred or transmitted to the expandable components 206. Expansion of the blowing agents 210 can cause the expandable components 206 (e.g., the thermoplastic microspheres) to expand or increase in volume. This ultimately causes the composite material 103 to expand or increase in volume.

The composite material 103 can expand or increase in size in an isotropic manner such that the composite material 103 expands in all directions. Such isotropic expansion can be harnessed to produce expansion or material displacement in specific directions by placing or positioning the composite material 103 at specific locations within the optic portion 102 of the adjustable IOL 100.

As previously discussed, the external energy can be laser light and the energy absorbing constituent 204 can absorb or capture the laser light directed at the composite material 103 and transform or transfer the light energy into thermal energy or heat to the expandable components 206. The blowing agent 210 within the expandable components 206 can expand or become energized in response to the thermal energy or heat. The expandable components 206 and, ultimately, the composite material 103 can expand or increase in volume in response to this light energy directed at the composite material 103.

The shape change (e.g., increase in volume) undertaken by the expandable components can be a persistent or a substantially permanent change. A persistent or substantially permanent change can mean that the expandable components 206 do not substantially revert back to its original shape or size after the shape change (e.g., after an increase in volume) has occurred. As a result, any change in the size or volume of the composite material 103 caused by a change in the size or volume of the expandable components 206 is also persistent or substantially permanent. As will be discussed in more detail in the following sections, this means that any structural changes made to the IOL 100 as a result of external energy or stimulus applied or otherwise directed at the composite material 103 embedded or integrated within the IOL 100 can persist or remain substantially permanent.

The thermoplastic shells 208 of the expandable components 206 can harden, once again, when the external energy is no longer directed or applied to the composite material 103. For example, the thermoplastic shells 208 may again harden when the temperature within a vicinity of the expandable components 206 falls below a certain threshold. For example, the thermoplastic shells 208 of the expandable microspheres can harden when light energy is no longer directed at the composite material 103. After the thermoplastic shells 208 harden, the expandable components 206 are locked into their new size and expanded configuration.

When the energy absorbing constituent 204 is an energy absorbing colorant, such as a dye or graphitized carbon, the color of at least part of the composite material 103 can take on the color of the energy absorbing colorant. For example, when the energy absorbing constituent 204 is an azo dye such as Disperse Red 1 having a red color, at least a portion of the composite material 103 comprising the energy absorbing constituent 204 can be colored red. Moreover, when the energy absorbing constituent 204 is graphitized carbon having a black color, at least a portion of the composite material 103 comprising the energy absorbing constituent 204 can be colored black. Although two colors (e.g., red and black) are mentioned in this disclosure, it is contemplated by this disclosure and it should be understood by one of ordinary skill in the art that energy absorbing colorant of other types of colors can also be used such as energy absorbing yellow, orange, or blue dyes or materials.

The color of the energy absorbing colorant can be visually perceptible to a clinician or another medical professional when at least part of the IOL 100 is made of the composite material 103 comprising the energy absorbing colorant. The color of the energy absorbing colorant can be visually perceptible to a clinician or another medical professional when the IOL 100 is implanted within an eye of a patient. For example, the composite material 103 can comprise Disperse Red 1 serving as the energy absorbing colorant. In this example, at least part of the IOL 100 can appear red or reddish to the clinician or another medical professional when the IOL 100 is implanted within the eye of a patient. The color of the energy absorbing colorant can allow the clinician or another medical professional to detect or determine the location or position of the composite material 103 within the IOL 100. The color of the energy absorbing colorant can also allow the clinician or another medical professional to determine where to direct the external energy to adjust the IOL 100.

One technical problem faced by the applicant is how to integrate the composite material 103 into the optic portion 102 of the adjustable IOL 100 such that the composite material 103 would adhere to the material used to make the rest of the adjustable IOL 100 and remain substantially fixed at certain locations within the optic portion 102. One solution discovered by the applicant and disclosed herein is the unique composition of the composite material 103 which incorporates the same copolymer blend used to make the rest of the optic portion 102. By designing the adjustable IOL 100 in this manner, the composite material 103 can be compatible with the material used to construct the rest of the optic portion 102 and can remain substantially fixed at its location without migrating or shifting.

Another technical problem faced by the applicant is how to ensure that any adjustments made to the adjustable IOL 100 persist long after the adjustment procedure. One solution discovered by the applicant and disclosed herein is to induce an expansion of a composite material 103 made in part of expandable microspheres comprising a blowing agent contained within thermoplastic shells. The thermoplastic shells can soften (and the thickness of the thermoplastic shells can decrease) in response to an external energy directed or applied at the composite material 103 (which can result in heat or thermal energy being transferred or transmitted to the expandable microspheres). The blowing agent within the thermoplastic shells can expand as the thermoplastic shells soften. Expansion of the blowing agent can expand the microspheres, which can, in turn, expand the composite base material serving as the bulk of the composite material 103. The expandable microspheres can retain their new enlarged or expanded configuration even after the external energy is no longer applied to the composite material 103.

Moreover, the energy absorbing constituent of the composite material 103 can capture or absorb a relatively harmless amount of external energy or stimulus directed at the composite material 103 and transform or transfer the external energy into thermal energy which can then cause the thermoplastic microspheres to expand. By designing the optic portion 102 of the adjustable IOL 100 in this manner, a burst of relatively harmless energy or stimulus (e.g., light energy) can be used to induce a persistent change in the shape or size of at least part of the optic portion 102. This persistent change in the shape or size of the optic portion 102 can have a continuing effect on an optical parameter of the lens including, for example, its base power.

FIG. 3 illustrates a cross-sectional view of one embodiment of the optic portion 102 of the adjustable IOL 100 shown in FIG. 1 in an assembled configuration. In this view, the haptics 104 of the adjustable IOL 100 have been removed for ease of viewing.

As shown in FIG. 3, the posterior element 108 can comprise a first posterior portion 112 and a second posterior portion 114. The first posterior portion 112 and the second posterior portion 114 can be made of the composite material 103.

In some embodiments, the second posterior portion 114 can be located radially inward from the first posterior portion 112. In these and other embodiments, the second posterior portion 114 can also be located posterior of the first posterior portion 112.

As shown in FIG. 3, the posterior element 108 can comprise an outer posterior surface 116, an inner posterior surface 118 facing the optic chamber 110, and a raised periphery 120. At least part of the inner posterior surface 118 can serve as a chamber floor 122 of the optic chamber 110. A sloped portion 124 of the inner posterior surface 118 can serve as a part of a chamber wall 126 surrounding the optic chamber 110.

In some embodiments, the first posterior portion 112 can be set within the chamber wall 126 and the second posterior portion 114 can be set within the chamber floor 122. In these and other embodiments, at least part of the chamber floor 122 of the optic portion 102 can be made of the composite material 103 and at least part of the chamber wall 126 of the optic portion 102 can be made of the composite material 103.

As shown in FIGS. 1 and 3, in certain embodiments, the first posterior portion 112 can be implemented in the form of a first ring-shaped segment 128 (see FIG. 1) and the second posterior portion 114 can be implemented in the form of a second ring-shaped segment 130 (see FIG. 1). The second ring-shaped segment 130 can be smaller in diameter than the first ring-shaped segment 128. The second ring-shaped segment 130 can be positioned concentric with the first ring-shaped segment 128.

In some embodiments, the first ring-shaped segment 128 (the first posterior portion 112) and the second ring-shaped segment 130 (the second posterior portion 114) can be connected together or implemented as a large outer ring with a stepped-down smaller inner ring connected to the large outer ring.

In other embodiments, the first ring-shaped segment 128 (the first posterior portion 112) can be disconnected from the second ring-shaped segment 130 (the second posterior portion 114) or spaced apart from the second ring-shaped segment 130 (the second posterior portion 114).

As will be discussed in more detail in the following sections, the base power of the optic portion 102 can be configured to decrease in response to an external energy (e.g., light energy) directed at the first posterior portion 112. Additionally, or alternatively, the base power of the optic portion 102 can be configured to increase in response to the external energy directed at the second posterior portion 114.

The first posterior portion 112 can be configured to expand in response to the external energy directed at the first posterior portion 112. A volume of the optic chamber 110 can increase in response to the expansion of the first posterior portion 112.

The second posterior portion 114 can be configured to expand in response to the external energy directed at the second posterior portion 114. The volume of the optic chamber can decrease in response to the expansion of the second posterior portion 114. For example, at least part of the second posterior portion 114 can be configured to expand in an anterior direction (into the optic chamber 110) in response to the external energy directed at the second posterior portion 114.

When the second posterior portion 114 is set within the chamber floor 122, the base power of the optic portion 102 can be configured to increase in response to the external energy directed at the chamber floor 122.

When the first posterior portion 112 is set within the chamber wall 126, the base power of the optic portion 102 can be configured to decrease in response to the external energy directed at the chamber wall 126.

One technical problem faced by the applicant is how to design an IOL that can be adjusted post-operatively by a clinician or other medical professional. One technical solution discovered by the applicant is the adjustable IOL 100 disclosed herein where the optic chamber 110 of the adjustable IOL 100 is filled with a fluid (e.g., a silicone oil) and at least part of the posterior element 108 of the optic portion 102 is made of a composite material 103. The base power of the optic portion 102 can be configured to change in response to an external energy directed at the composite material 103.

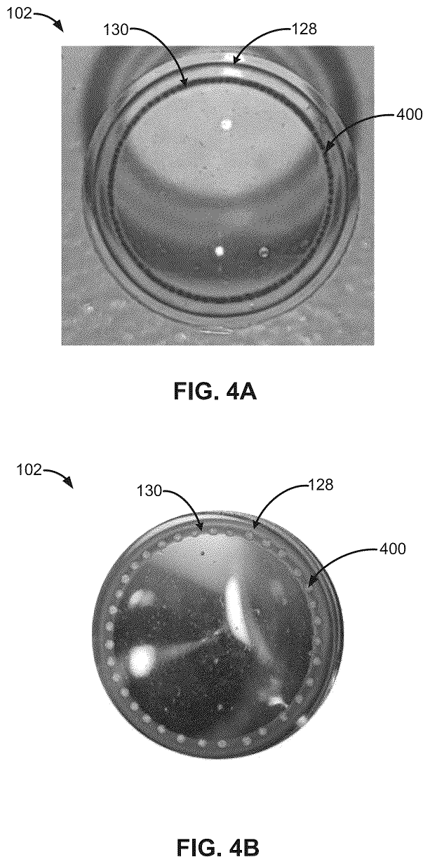

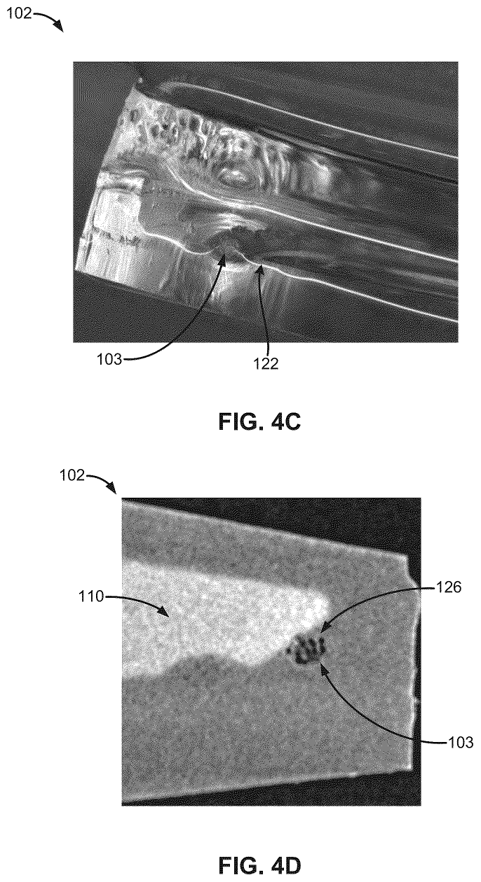

FIG. 4A is a black-and-white image of the optic portion 102 of one embodiment of the adjustable IOL 100 after pulses of laser light (the external energy in this case) have been directed at the composite material 103 making up a part of the optic portion 102.

As shown in FIG. 4A, the optic portion 102 can comprise a first ring-shaped segment and a second ring-shaped segment 130. As shown in FIG. 4A, the second ring-shaped segment 130 can be smaller in diameter than the first ring-shaped segment 128. Moreover, the second ring-shaped segment 130 can be positioned concentric with the first ring-shaped segment 128.

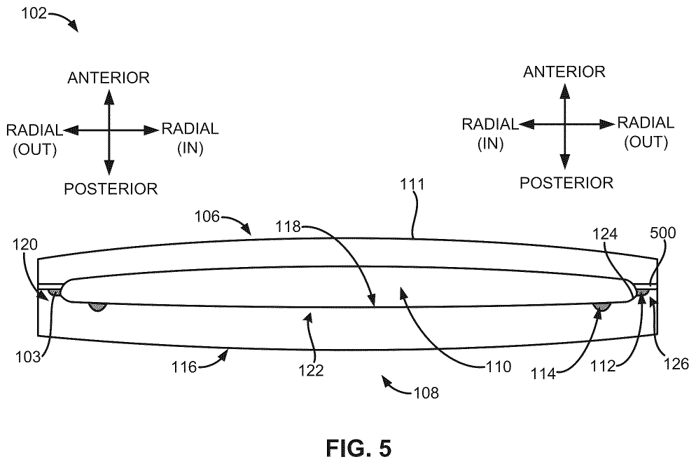

Furthermore, the first ring-shaped segment 128 can be disconnected or spaced apart from the second ring-shaped segment 130 (see, also, FIG. 5).

As shown in FIG. 4A, pulses of laser light have been directed at numerous target sites 400 around the second ring-shaped segment 130 made of the composite material 103. The target sites 400 that have received the laser light have expanded and appear as substantially-circular dots when viewed from a top-down perspective.

In this instance, the volume of the optic chamber 110 can be configured to decrease in response to the expansion of the composite material 103 at the target sites 400. As such, the fluid pressure within the optic chamber 110 can increase and cause the anterior element 106 to deform, flex, or otherwise change shape. This shape change can cause the base power of the optic portion 102 to increase.

FIG. 4B is a black-and-white image of an optic portion 102 of another embodiment of the adjustable IOL 100 after pulses of laser light (the external energy in this case) have been directed at the composite material 103 making up a part of the optic portion 102. For example, the optic portion 102 shown in FIG. 4B can be the assembled version of the optic portion 102 depicted in FIG. 1 and/or the optic portion depicted in FIG. 3.

The optic portion 102 can comprise a first ring-shaped segment 128 and a second ring-shaped segment 130. As shown in FIG. 4B, the second ring-shaped segment 130 can be smaller in diameter than the first ring-shaped segment 128. Moreover, the second ring-shaped segment 130 can be positioned concentric with the first ring-shaped segment 128.

Furthermore, the first ring-shaped segment 128 can be connected or otherwise coupled to the second ring-shaped segment 130. For example, the first ring-shaped segment 128 can be implemented as a large outer ring and the second ring-shaped segment 130 can be implemented as a smaller stepped-down inner ring connected to the large outer ring.

As shown in FIG. 4B, pulses of laser light have been directed at 35 target sites 400 around the second ring-shaped segment 130. The target sites 400 that have received the laser light have expanded and appear as discrete substantially-circular dots when viewed from a top-down perspective.

In this instance, the volume of the optic chamber 110 can be configured to decrease in response to the expansion of the composite material 103 at the target sites 400. As such, the fluid pressure within the optic chamber 110 can increase and cause the anterior element 106 to deform, flex, or otherwise change shape. This shape change can cause the base power of the optic portion 102 to increase.

FIG. 4C is a close-up black-and-white image showing an activated composite material 103 expanding into an optic chamber 110 in response to an external energy having been directed at the composite material 103. For example, the composite material 103 can be a second posterior portion 114 set within a chamber floor 122 of the optic portion 102.

When the composite material 103 is activated by receiving the external energy (e.g., pulses of laser light), at least part of the composite material 103 can rise up from the chamber floor 122 and expand in an anterior direction into the optic chamber 110. This expansion of the composite material 103 can decrease the volume of the optic chamber 110. As a result, the base power of the optic portion 102 can be configured to increase in response to the external energy directed at the chamber floor 122.

FIG. 4D is a computed tomography (CT) scan showing an activated composite material 103 making up part of a chamber wall 126 surrounding an optic chamber 110 of the optic portion 102. For example, the composite material 103 can be a first posterior portion 112 set within the chamber wall 126.

When the composite material 103 is activated by receiving the external energy (e.g., pulses of laser light), at least part of the composite material 103 can push out (for example, in a radially outward direction) the chamber wall 126. This expansion of the composite material 103 can increase a volume of the optic chamber 110. As a result, the base power of the optic portion 102 can be configured to decrease in response to the external energy directed at the chamber wall 126.

FIG. 5 illustrates a cross-sectional view of the optic portion 102 of another embodiment of the adjustable IOL 100. As shown in FIG. 5, the posterior element 108 can comprise a first posterior portion 112 and a second posterior portion 114. The first posterior portion 112 and the second posterior portion 114 can be made of the composite material 103.

In some embodiments, the second posterior portion 114 can be located radially inward from the first posterior portion 112. In these and other embodiments, the second posterior portion 114 can also be located posterior of the first posterior portion 112.

As shown in FIG. 5, the posterior element 108 can comprise an outer posterior surface 116, an inner posterior surface 118 facing the optic chamber 110, and a raised periphery 120.

At least part of the inner posterior surface 118 can serve as a chamber floor 122 of the optic chamber 110. A sloped portion 124 of the inner posterior surface 118 can serve as a part of a chamber wall 126 surrounding the optic chamber 110.

In some embodiments, the first posterior portion 112 can be set within the chamber wall 126 and the second posterior portion 114 can be set within the chamber floor 122. In these and other embodiments, at least part of the chamber floor 122 of the optic portion 102 can be made of the composite material 103 and at least part of the chamber wall 126 of the optic portion 102 can be made of the composite material 103.

As shown in FIGS. 4A and 5, in certain embodiments, the first posterior portion 112 can be implemented in the form of a first ring-shaped segment 128 (see FIG. 4A) and the second posterior portion 114 can be implemented in the form of a second ring-shaped segment 130 (see FIG. 4A). The second ring-shaped segment 130 can be smaller in diameter than the first ring-shaped segment 128. The second ring-shaped segment 130 can be positioned concentric with the first ring-shaped segment 128.

In these embodiments, the first ring-shaped segment 128 (the first posterior portion 112) can be disconnected from the second ring-shaped segment 130 (the second posterior portion 114) or spaced apart from the second ring-shaped segment 130 (the second posterior portion 114).

As will be discussed in more detail in the following sections, the base power of the optic portion 102 can be configured to decrease in response to an external energy (e.g., light energy) directed at the first posterior portion 112. Additionally, or alternatively, the base power of the optic portion 102 can be configured to increase in response to the external energy directed at the second posterior portion 114.

The first posterior portion 112 can be configured to expand in response to the external energy directed at the first posterior portion 112. Expansion of the first posterior portion 112 can increase the height of the optic chamber 110 by pushing up against an interface 500 between the anterior element 106 and the posterior element 108. In some embodiments, the interface 500 can be a glue gap or adhesive layer. As a result, a volume of the optic chamber 110 can increase in response to the expansion of the first posterior portion 112.

Moreover, expansion of the first posterior portion 112 can also push out the chamber wall 126 such that the optic chamber 110 is expanded radially. In this instance, the volume of the optic chamber 110 is also increased.

In the embodiment shown in FIG. 5, the first posterior portion 112 (made of the composite material 103) can be positioned slightly posterior of the glue gap or adhesive layer (shown as the interface 500 in FIG. 5) but is not part of the glue gap or adhesive layer. In this embodiment, the adhesive layer or interface 500 can be separate from the first posterior portion 112.

The second posterior portion 114 can be configured to expand in response to the external energy directed at the second posterior portion 114. The volume of the optic chamber 110 can decrease in response to the expansion of the second posterior portion 114. For example, at least part of the second posterior portion 114 can be configured to expand in an anterior direction (into the optic chamber 110) in response to the external energy directed at the second posterior portion 114.

When the second posterior portion 114 is set within the chamber floor 122, the base power of the optic portion 102 can be configured to increase in response to the external energy directed at the chamber floor 122.

When the first posterior portion 112 is set within part of the chamber wall 126, the base power of the optic portion 102 can be configured to decrease in response to the external energy directed at the chamber wall 126.

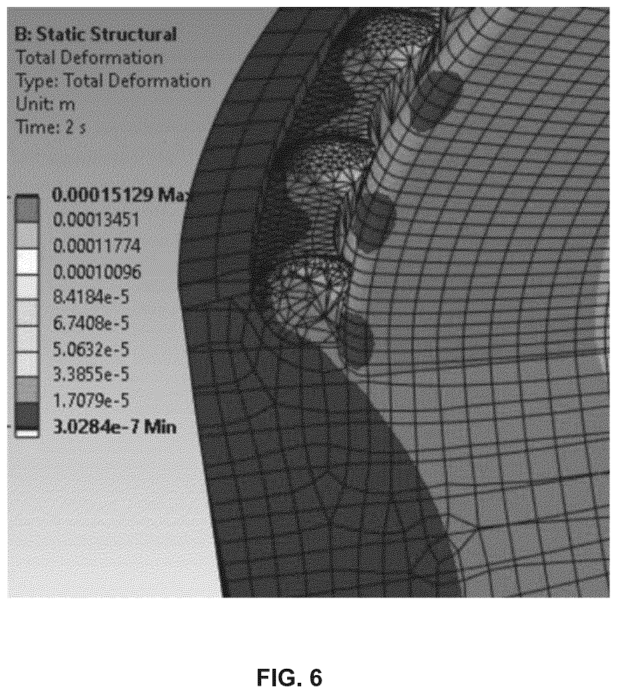

FIG. 6 is an image showing the results of a finite element analysis (FEA) performed on an embodiment of the adjustable IOL 100 shown in FIG. 5 after pulses of laser light are directed at the first posterior portion 112 of the adjustable IOL 100. As shown in FIG. 6, the pulses of laser light can be directed at multiple target sites along the first posterior portion 112 making up part of the chamber wall 126.

At each target site, the composite material 103 making up the first posterior portion 112 can expand in a substantially spherical manner. This expansion can push out the chamber wall 126 such that the optic chamber 110 is expanded radially. In this instance, the volume of the optic chamber 110 is increased. As a result, the fluid pressure within the optic chamber 110 decreases and the base power of the optic portion 102 decreases.

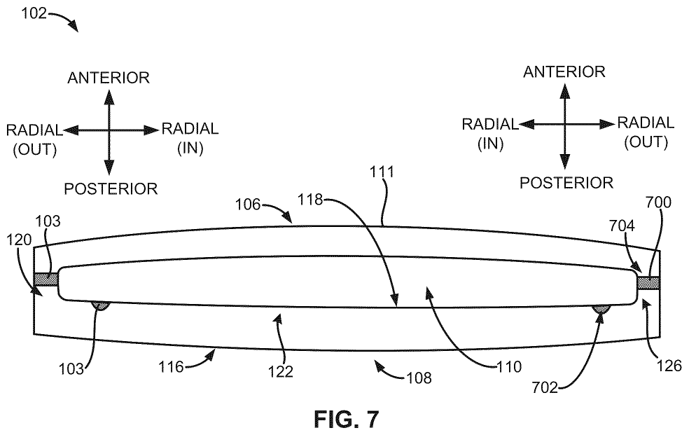

FIG. 7 illustrates a cross-sectional view of the optic portion 102 of yet another embodiment of the adjustable IOL 100 where an adhesive layer 700 comprises the composite material 103. The anterior element 106 can be bonded or adhered to the posterior element 108 by the adhesive layer 700.

As shown in FIG. 7, the posterior element 108 can comprise a posterior portion 702 made of the composite material 103. The posterior portion 702 can be located radially inward from a radial position of the adhesive layer 700. The posterior portion 702 can also be located posterior of the adhesive layer 700.

As shown in FIG. 7, the posterior element 108 can comprise an outer posterior surface 116, an inner posterior surface 118 facing the optic chamber 110, and a raised periphery 120. At least part of the inner posterior surface 118 can serve as a chamber floor 122 of the optic chamber 110. The raised periphery 120 can serve as a part of a chamber wall 126 surrounding the optic chamber 110.

The adhesive layer 700 can be located at an interface or contact layer between the anterior element 106 and the posterior element 108. For example, the adhesive layer 700 can be located in between the raised periphery 120 of the posterior element 108 and a radially-outer periphery 704 of the anterior element 106. For example, the adhesive layer 700 can be implemented in the form of a first ring-shaped segment.

In some embodiments, the posterior portion 702 can be set within the chamber floor 122. In these and other embodiments, at least part of the chamber floor 122 of the optic portion 102 can be made of the composite material 103. For example, the posterior portion 702 can be implemented in the form of a second ring-shaped segment smaller in diameter than the first ring-shaped segment. The second ring-shaped segment can be positioned concentric with the first ring-shaped segment.

In some embodiments, the first ring-shaped segment can be disconnected from the second ring-shaped segment or spaced apart from the second ring-shaped segment.

The base power of the optic portion 102 can be configured to decrease in response to an external energy (e.g., light energy) directed at the adhesive layer 700. Additionally, or alternatively, the base power of the optic portion 102 can be configured to increase in response to the external energy directed at the posterior portion 702.

The adhesive layer 700 can be configured to expand in response to the external energy directed at the adhesive layer 700. The posterior portion 702 can be configured to expand in response to the external energy directed at the posterior portion 702. Expansion of the adhesive layer 700 can increase the height of the optic chamber 110 by pushing up against the anterior element 106 and pushing down against the posterior element 108. The volume of the optic chamber 110 can increase in response to the expansion of the adhesive layer 700. As a result, the base power of the optic portion 102 can be configured to decrease in response to the external energy directed at the adhesive layer 700.

In some embodiments, the adhesive layer 700 can also be referred to as a glue gap or glue layer.

The posterior portion 702 can be configured to expand in response to the external energy directed at the posterior portion 702. The volume of the optic chamber 110 can decrease in response to the expansion of the posterior portion 702. For example, at least part of the posterior portion 702 can be configured to expand in an anterior direction (into the optic chamber 110) in response to the external energy directed at the posterior portion 702.

When the posterior portion 702 is set within the chamber floor 122, the base power of the optic portion 102 can be configured to increase in response to the external energy directed at the chamber floor 122.

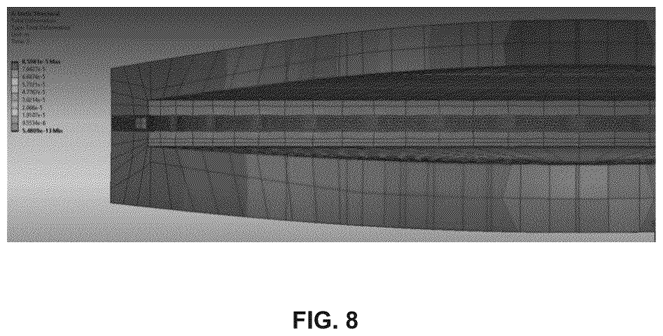

FIG. 8 is an image showing the results of a finite element analysis (FEA) performed on the adjustable IOL 100 of FIG. 7 after pulses of laser light are directed at the adhesive layer 700 made in part of the composite material 103.

As shown in FIG. 8, the pulses of laser light can be directed at multiple target sites along the adhesive layer 700. At each target site, the composite material 103 making up parts of the adhesive layer 700 can expand in a substantially spherical manner. This expansion can push the posterior element 108 further away from the anterior element 106 such that the volume of the optic chamber 110 is increased. As a result, the fluid pressure within the optic chamber 110 decreases and the base power of the optic portion 102 decreases.

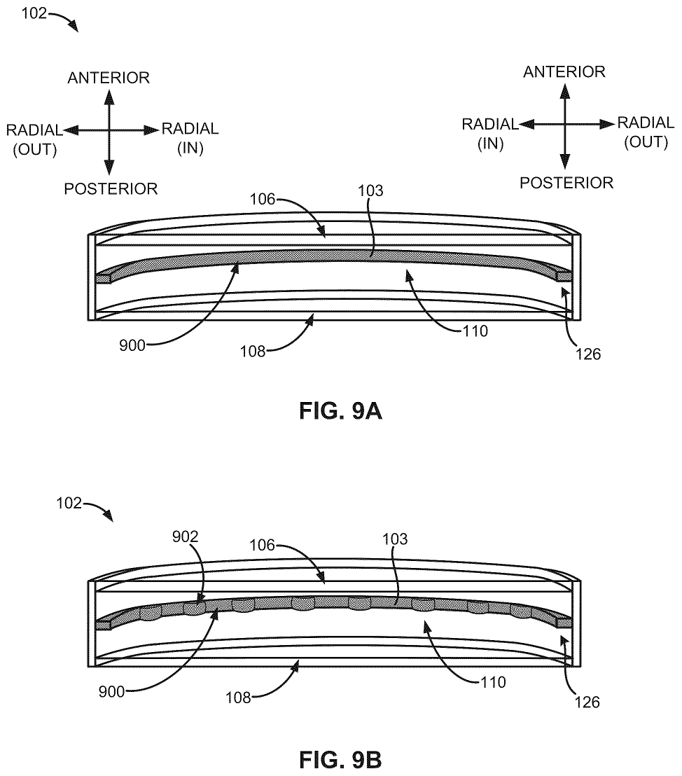

FIG. 9A illustrates a cross-sectional view of an optic portion 102 of one embodiment of the adjustable IOL 100 with the composite material 103 extending radially inward from a chamber wall 126 of the adjustable IOL 100.

As shown in FIGS. 9A and 9B, the composite material 103 can be implemented as a circular rim 900 extending radially inward from the chamber wall 126 surrounding the optic chamber 110.

FIG. 9B illustrates a cross-sectional view of the optic portion 102 of the adjustable IOL of FIG. 9A after pulses of laser light are directed at the composite material 103 (implemented as the circular rim 900) extending radially inward from the chamber wall 126 of the adjustable IOL 100.

As shown in FIG. 9B, the pulses of laser light can be directed at multiple target sites 902 along the circular rim 900. At each target site 902, the composite material 103 making up parts of the circular rim 900 can expand in a substantially spherical or rotund manner. This expansion can cause portions of the circular rim 900 to extend radially inward into the optic chamber 110. As a result, the volume of the optic chamber 110 can decrease and the fluid pressure within the optic chamber 110 can increase. This can result in an increase in the base power of the optic portion 102.

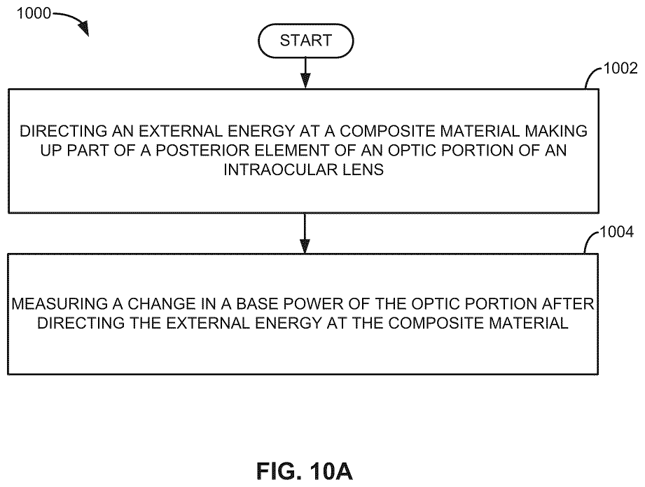

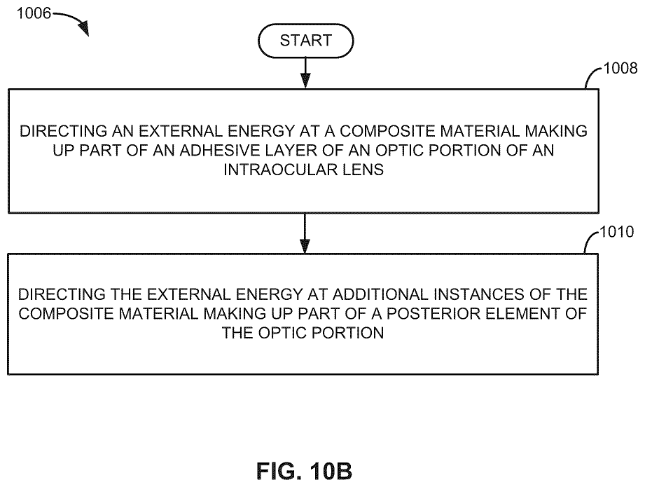

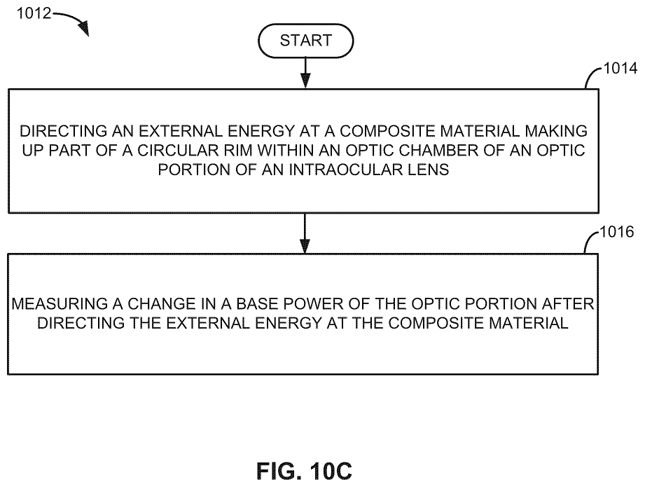

FIG. 10A is one embodiment of a method 1000 of adjusting an IOL post-operatively or post-implantation. The method 1000 can comprise directing an external energy at a composite material making up part of a posterior element of an optic portion of the IOL in operation 1002.

The optic portion can also comprise an anterior element and an optic chamber defined in between the anterior element and the posterior element. The optic chamber can be filled with a fluid. A base power of the optic portion can be configured to change in response to the external energy directed at the composite material.

For example, operation 1002 can comprise directing the external energy at a first posterior portion within a chamber wall of the optic portion in order to decrease the base power of the optic portion. Also, for example, operation 1002 can comprise directing the external energy at a second posterior portion within a chamber floor in order to increase the base power of the optic portion.

The method 1000 can also comprise measuring the change in the base power of the optic portion after directing the external energy at the composite material in operation 1004.

FIG. 10B is one embodiment of another method 1006 of adjusting an IOL post-operatively or post-implantation. The method 1000 can comprise directing an external energy at a composite material making up part of an adhesive layer of an optic portion of the IOL in operation 1008. The adhesive layer can be configured to expand in response to the external energy directed at the adhesive layer. Expansion of the adhesive layer can increase a volume of the optic chamber. A base power of the optic portion can be configured to decrease in response to the external energy directed at the composite material making up part of the adhesive layer

The method 1006 can also comprise directing an external energy at additional instances of the composite material making up part of a posterior element of the optic portion of the IOL in operation 1010.

For example, operation 1010 can comprise directing the external energy at a first posterior portion within a chamber wall of the optic portion in order to further decrease the base power of the optic portion. Also, for example, operation 1010 can comprise directing the external energy at a second posterior portion within a chamber floor in order to increase the base power of the optic portion.

FIG. 10C is one embodiment of another method 1012 of adjusting an IOL post-operatively or post-implantation. The method 1012 can comprise directing an external energy at a composite material making up part of a circular rim within an optic chamber of an optic portion of the IOL.

At least part of the circular rim can be configured to expand in a radially inward direction in response to the external energy directed at the circular rim. Expansion of the circular rim can decrease a volume of the optic chamber and increase the base power of the optic portion.

The method 1012 can further comprise measuring the change in the base power of the optic portion after directing the external energy at the composite material in operation 1016.

In one or more of the methods disclosed herein, directing the external energy at the composite material can comprise directing laser light having a wavelength between about 488 nm to about 650 nm at the composite material. In other embodiments, directing the external energy at the composite material can further comprise directing laser light having a wavelength between about 946 nm to about 1120 nm at the composite material.

One drawback of currently available tunable IOLs (such as light adjustable lens) is that the tuning procedure requires time to take effect, may require multiple visits to a clinician's office, and the clinician must often purchase expensive new equipment to undertake such tuning procedures.

One advantage of the adjustable IOLs 100 disclosed herein is that such IOLs allow for post-operative refractive error correction in a matter of seconds rather than hours. This allows patients to provide feedback concerning their refractive error correction almost instantaneously. Moreover, the IOLs 100 disclosed herein can be tuned using commercially available lasers (e.g., 532 nm photocoagulator lasers) that are commonly found in most clinician's offices.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. An adjustable intraocular lens, comprising:
an optic portion, comprising:
an anterior element,
a posterior element, and
an optic chamber defined therebetween,
wherein the optic chamber is filled with a fluid,
wherein at least part of the posterior element is made of a composite material, and
wherein a base power of the optic portion is configured to change in response to an external energy directed at the composite material.

2. The adjustable intraocular lens of claim 1, wherein the posterior element comprises a first posterior portion and a second posterior portion, and wherein the first posterior portion and the second posterior portion are made of the composite material.

3. The adjustable intraocular lens of claim 2, wherein the second posterior portion is located radially inward from the first posterior portion.

4. The adjustable intraocular lens of claim 2, wherein the second posterior portion is located posterior of the first posterior portion.

5. The adjustable intraocular lens of claim 2, wherein the base power of the optic portion is configured to decrease in response to the external energy directed at the first posterior portion.

6. The adjustable intraocular lens of claim 5, wherein the first posterior portion is configured to expand in response to the external energy directed at the first posterior portion, and wherein expansion of the first posterior portion increases a volume of the optic chamber.

7. The adjustable intraocular lens of claim 2, wherein the base power of the optic portion is configured to increase in response to the external energy directed at the second posterior portion.

8. The adjustable intraocular lens of claim 7, wherein the second posterior portion is configured to expand in response to the external energy directed at the second posterior portion, and wherein expansion of the second posterior portion decreases a volume of the optic chamber.

9. The adjustable intraocular lens of claim 2, wherein the first posterior portion is in the form of a first ring-shaped segment, and wherein the second posterior portion is in the form of a second ring-shaped segment.

10. The adjustable intraocular lens of claim 9, wherein the second ring-shaped segment is smaller in diameter than the first ring-shaped segment.

11. The adjustable intraocular lens of claim 9, wherein the second ring-shaped segment is positioned concentric with the first ring-shaped segment and radially inward of the first ring-shaped segment.

12. The adjustable intraocular lens of claim 2, wherein the posterior element comprises an outer posterior surface and an inner posterior surface facing the optic chamber, and wherein part of the inner posterior surface serves as a chamber floor of the optic chamber.

13. The adjustable intraocular lens of claim 12, wherein the second posterior portion is set within the chamber floor.

14. The adjustable intraocular lens of claim 13, wherein part of the second posterior portion is configured to expand in an anterior direction in response to the external energy directed at the second posterior portion.

15. The adjustable intraocular lens of claim 12, wherein a sloped portion of the inner posterior surface serves as part of a chamber wall of the optic chamber.

16. The adjustable intraocular lens of claim 15, wherein the first posterior portion is set within the sloped portion of the inner posterior surface.

17. The adjustable intraocular lens of claim 16, wherein part of the first posterior portion is configured to expand in a radially outward direction in response to the external energy directed at the first posterior portion.

18.-36. (canceled)

37. A method of adjusting an intraocular lens post-implantation, comprising:
directing an external energy at a composite material making up part of a posterior element of an optic portion of the intraocular lens, wherein the optic portion further comprises:
an anterior element, and
an optic chamber defined in between the anterior element and the posterior element,
wherein the optic chamber is filled with a fluid, and
wherein a base power of the optic portion is configured to change in response to the external energy directed at the composite material; and
measuring the change in the base power of the optic portion after directing the external energy at the composite material.

38.-71. (canceled)

72. An adjustable intraocular lens, comprising:
an optic portion, comprising an optic chamber filled with a fluid, wherein the optic chamber comprises:
a chamber floor, and
chamber walls,
wherein at least part of the chamber floor is made of a composite material, and
wherein a base power of the optic portion is configured to change in response to an external energy directed at the chamber floor.

73.-75. (canceled)

76. An adjustable intraocular lens, comprising:
an optic portion, comprising:
an optic chamber filled with a fluid,
wherein at least part of the optic chamber is made of a composite material, and
wherein a base power of the optic portion is configured to change in response to an external energy directed at the composite material.

77.-107. (canceled)
